(19) United States
(12) Patent Application Publication
(10) Pub. No.: US 2024/0225906 A1
(43) Pub. Date: Jul. 11, 2024

(54) CBD-INFUSED THERAPY TAPE AND DISPENSER

(71) Applicants: Rodney Shaw, Mesa, AZ (US); STEPHANIE MITCHELL-ANTHONY, Mesa, AZ (US); KEVIN HARRIS, Mesa, AZ (US); PEGGY EDWARDS-HARRIS, Mesa, AZ (US)

(72) Inventors: Rodney Shaw, Mesa, AZ (US); STEPHANIE MITCHELL-ANTHONY, Mesa, AZ (US); KEVIN HARRIS, Mesa, AZ (US); PEGGY EDWARDS-HARRIS, Mesa, AZ (US)

(21) Appl. No.: 18/093,785

(22) Filed: Jan. 5, 2023

(51) Int. Cl. A61F 13/00; A61F 13/02; A61F 15/00; A61K 31/05

(52) U.S. Cl. CPC A61F 13/00063; A61F 13/0283; A61F 15/002; A61K 31/05; A61F 2013/0296

(57) ABSTRACT

A method and apparatus of a CBD-infused therapy tape and dispenser device for ease and mobile convenience for a person requiring the need of CBD for medical purposes. The device has a tape where the tape has a middle, an interior surface and an exterior surface. The tape is infused with cannabidiol (CBD) solution and a core where the core being in a middle of a tape roll when the tape is rolled around the core. And a method of making a CBD-infused tape roll by first unrolling the tape roll where the unrolled tape roll presents an interior surface and formulating a CBD solution. Then, using an applicator to applying the CBD solution onto the raw tape roll onto the interior surface of the unrolled tape roll.

### FIELD OF THE INVENTION

This invention relates to CBD dispensing devices. More particularly, it relates to an therapy device for CBD infusing utilizing a tape and dispenser.

### BACKGROUND

Cannabidiol (CBD) is a Phyto-cannabinoid discovered in 1940. It is one of 113 identified cannabinoids in cannabis plants, along with tetrahydrocannabinol (THC), and accounts for up to 40% of the plant's extract. As of 2019, clinical research on CBD included studies related to anxiety, cognition, movement disorders, and pain, but there is insufficient high-quality evidence that cannabidiol is effective for these conditions. Nevertheless, CBD is a popular herbal drug, widely promoted with claims of particular therapeutic benefits.

Cannabidiol can be taken internally in multiple ways, including by inhaling cannabis smoke or vapor, by mouth, and as an aerosol spray into the cheek. It may be supplied as CBD oil containing only CBD as the active ingredient (excluding tetrahydrocannabinol [THC] or terpenes), CBD-dominant hemp extract oil, capsules, dried cannabis, or prescription liquid solution. CBD does not have the same psychoactivity as THC, and will negate the psychoactive effects of THC on the body if both are present. As of 2018, the mechanism of action for its biological effects has not been determined. Unlike THC, which acts on the cannabinoid receptor type 1 (CB1) as a partial agonist, CBD instead is a negative allosteric modulator of CB1 receptors.

However the current methods for providing CBD to patients requiring and needing of the beneficial aspects of CBD are currently cumbersome and/or provide an odor for others to know of the use of a medication for their medical issues. Furthermore, the mechanisms for dispensing CBD require means sometimes not necessarily convenient to a patient.

Accordingly, and in light of the foregoing, there is a need for a device that can provide a quick, safe and mobility of CBD for use by those requiring the medical benefits of CBD. The device should be easy to use, cost-efficient and easily transported.

### BRIEF DESCRIPTION OF THE DRAWINGS

The advantages and features of the present invention will become better understood with reference to the following more detailed description and claims taken in conjunction with the accompanying drawings, in which like elements are identified with like symbols, and in which:

FIG. 1 is a perspective view of the tape roll 15 as used with the CBD-infused therapy tape-and-dispenser 10, according to the preferred embodiment of the present invention;

FIG. 2 is a pictorial view of the process used to manufacture the tape roll 15, according to the preferred embodiment of the present invention;

FIG. 3 is a side perspective view of a tape wrap dispenser 85, used with the CBD-infused therapy tape-and-dispenser 10, according to the preferred embodiment of the present invention;

FIG. 4 is a front view of the tape wrap dispenser 85, used with the CBD-infused therapy tape-and-dispenser 10, according to the preferred embodiment of the present invention;

FIG. 5 is an exploded view of the tape wrap dispenser 85, used with the CBD-infused therapy tape-and-dispenser 10, according to the preferred embodiment of the present invention;

FIG. 6 is a sectional view of the tape wrap dispenser 85, used with the CBD-infused therapy tape-and-dispenser 10, as seen along a line I-I, as shown in FIG. 4, according to the preferred embodiment of the present invention; and,

FIG. 7 is a side view of the tape wrap dispenser 85 having a decorative exterior motif, according to the preferred embodiment of the present invention.

### DESCRIPTIVE KEY

10 CBD-infused therapy tape-and-dispenser
15 tape roll
20 tape
25 core
30 exterior surface
35 interior surface
40 adhesive coating
45 raw tape roll
50 CBD solution
55 reservoir
60 tubing
65 spray mechanism
70 spray nozzle
75 aerosol spray
80 CBD monolayer
85 tape wrap dispenser
90 outer enclosure
95 left side face
100 center section face
105 right side face
110 tape exit slot
115 release/cut button
120 center aperture
125 neck lanyard
130 quick release mechanism
135 force lines “f”
140 cutting blade
145 recessed lip
150 locking tabs
155 central hub
160 linkage cable
165 tape holding cavity
170 decorative indicia

### DESCRIPTION OF THE PREFERRED EMBODIMENTS

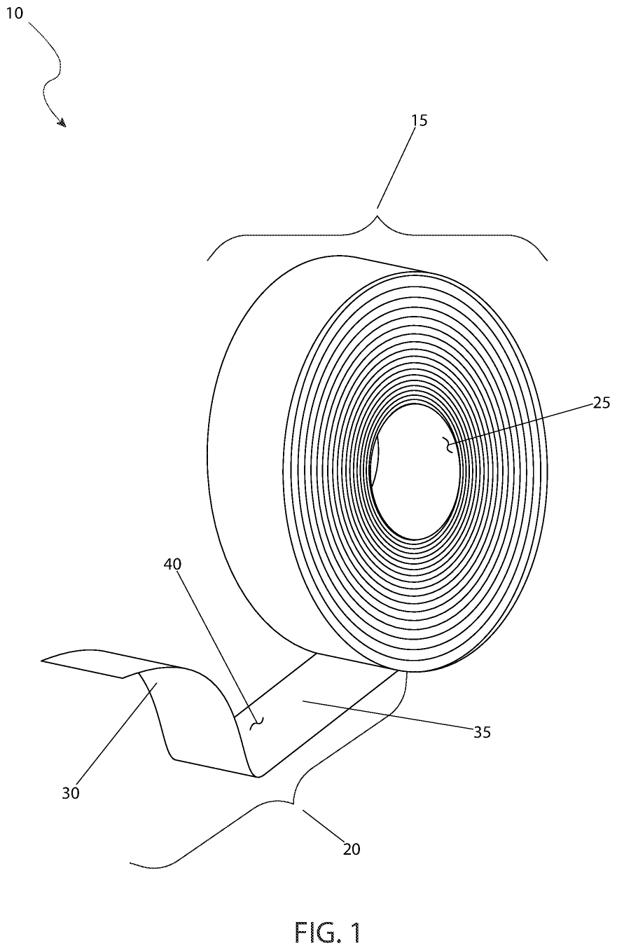

The best mode for carrying out the invention is presented in terms of its preferred embodiment, herein depicted within FIGS. 1 through 7. However, the invention is not limited to the described embodiment, and a person skilled in the art will appreciate that many other embodiments of the invention are possible without deviating from the basic concept of the invention and that any such work around will also fall under scope of this invention. It is envisioned that other styles and configurations of the present invention can be easily incorporated into the teachings of the present invention, and only one (1) particular configuration shall be shown and described for purposes of clarity and disclosure and not by way of limitation of scope. All of the implementations described below are exemplary implementations provided to enable persons skilled in the art to make or use the embodiments of the disclosure and are not intended to limit the scope of the disclosure, which is defined by the claims.

The terms “a” and “an” herein do not denote a limitation of quantity, but rather denote the presence of at least one (1) of the referenced items.

### DETAILED DESCRIPTION OF THE FIGURES

Referring now to FIG. 1, a perspective view of the tape roll 15, as used with a CBD-infused therapy tape-and-dispenser 10, according to the preferred embodiment of the present invention is disclosed. The CBD-infused therapy tape-and-dispenser 10 provides for a tape 20, preferably infused with cannabidiol (i.e., CBD) and configured in a tape roll 15, along with a method of assisted application such as that with a specialized tape wrap dispenser 85, that may be used to reduce discomfort due to pain. The CBD-infused therapy tape-and-dispenser 10 utilizes a CBD-infused tape roll 15 which produces a linear portion of CBD-infused tape 20 as a leading edge. The tape roll 15 is envisioned to be provided with a disposable core 25 upon which it is wound. The tape 20 is provided with an exterior surface 30, envisioned to be durable and water-resistant. The teachings of the tape 20 may be applied to virtually any type of tape 20 including athletic tape and more specifically kinesiology tape, envisioned to be made available in a wide variety of aesthetically pleasing colors. However, the use of any specific type of substrate used with the tape 20, shall not be interpreted as a limiting factor of the present invention. An interior surface 35 of the tape 20 is envisioned to be treated with a skin-safe adhesive coating 40 for application directly to human skin as would be typically expected.

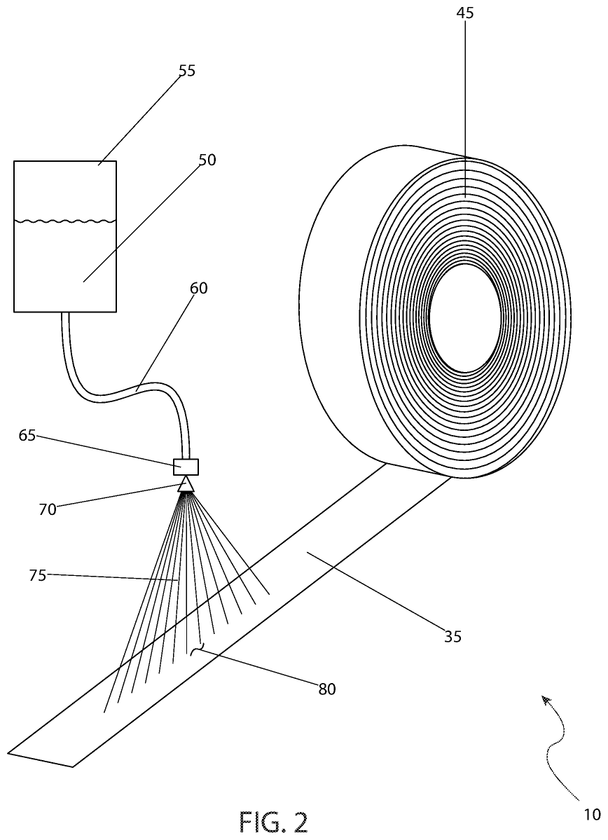

Referring next to FIG. 2, a pictorial view of the process used to manufacture the tape roll 15, according to the preferred embodiment of the present invention is depicted, during a manufacturing process. A raw tape roll 45 is unrolled such that it presents the interior surface 35 in an accessible position. A CBD solution 50, in at least an exemplary embodiment, comprises two grams (2 g) of CBD isolate completely dissolved in ninety-nine percent (99%) isopropyl alcohol produced in various concentrations to include, but not limited to: twenty-five milligrams per milliliter (25 mg/mL), fifty-seven-point-five milligrams per milliliter (57.5 mg/mL) and approximately one hundred milligrams per milliliter (˜100 mg/mL). Preferably, this CBD solution 50 is applied to the raw tape roll 45 in a roll-on-type of application by use of a roller ball or other suitable roll-on applicator. Alternately, the CBD solution 50 can be stored in a reservoir 55 and routed along tubing 60 to a spray mechanism 65, which includes a spray nozzle 70. The spray nozzle 70 thus produces an aerosol spray 75 which results in a CBD monolayer 80 on the interior surface 35. The use of a roll-on or aerosol application method shall not be interpreted as a limiting factor of the present invention.

The production of the twenty-five milligrams per milliliter (25 mg/mL) concentration is envisioned to proceed along the following process: use of five hundred milligrams (500 mg) CBD isolate; use of twenty milliliters (20 mL) isopropanol, allowed to mix and dissolve for fifteen minutes (15 min) at room temp; yield of twenty milliliters (20 mL) of a twenty-five milligram per milliliter (25 mg/mL) solution; application of one milliliter (1 mL) of twenty-five milligrams per milliliter (25 mg/mL) solution to each strip with syringe/aerosol spray; dry at room temp for fifteen minutes (15 min); resealed; the creation of seventeen (17) strips with twenty-five milligrams (25 mg) per strip; thus seventeen (17) strips of twenty-five milligrams (25 mg) of CBD per strip equates to four hundred twenty-five milligrams (425 mg) CBD used and (three milliliters (3 mL) or approximately seventy-five milligrams (75 mg) remaining from stock solution).

The production of the fifty-seven-point-five milligrams per milliliter (57.5 mg/mL) concentration is envisioned to proceed along the following process: use of five hundred milligrams (500 mg) CBD isolate; use of seven milliliters (7 mL) isopropanol; allowed to mix and dissolve for fifteen minutes (15 min) at room temperature, addition of remaining three milliliters (3 mL) or seventy-five milligrams (75 mg) from the production of the twenty-five milligrams per milliliter (25 mg/mL) concentration, as aforementioned described, for a total concentration of approximately five hundred seventy-five milligrams (575 mg) in ten milliliters (10 mL) isopropanol; yield of ten milliliters (10 mL) at fifty-seven-point-five milligrams per milliliter (57.5 mg/mL); application of one milliliter (1 mL) of fifty-seven-point-five milligrams per milliliter (57.5 mg/mL) solution to each strip with syringe/aerosol spray; dry at room temperature for fifteen minutes (15 min); resealed; the creation of ten (10) strips with fifty-seven-point-five milligrams (57.5 mg) per strip; thus, ten (10) strips of fifty-seven-point-five milligrams (57.5 mg) of CBD per strip equates to five hundred seventy-five milligrams CBD (575 mg) CBD used.

The production of approximately one hundred milligrams per milliliter (˜100 mg/mL) concentration is envisioned to proceed along the following process: use of one gram (1 g) CBD; use of ten milliliters (10 mL) isopropanol; allowed to mix and dissolve for fifteen minutes (15 min); yield of ten milliliters (10 ml) of a one hundred milligram per milliliter (100 mg/mL) solution; application of one milliliter (1 mL) of one hundred milligrams per milliliter (100 mg/mL) solution to each strip with syringe/aerosol spray; dry at room temp for fifteen minutes (15 min); resealed; the creation of ten (10) strips with approximately one hundred milligrams (100 mg) per strip; thus, ten (10) strips of one hundred milligrams (100 mg) CBD per strip equates to one gram (1 g) CBD used.

After a suitable period of drying, the tape 20 is rewound into a tape roll 15 to produce the CBD-infused tape roll 15 (as shown in FIG. 1) and packaged in a suitable manner for distribution.

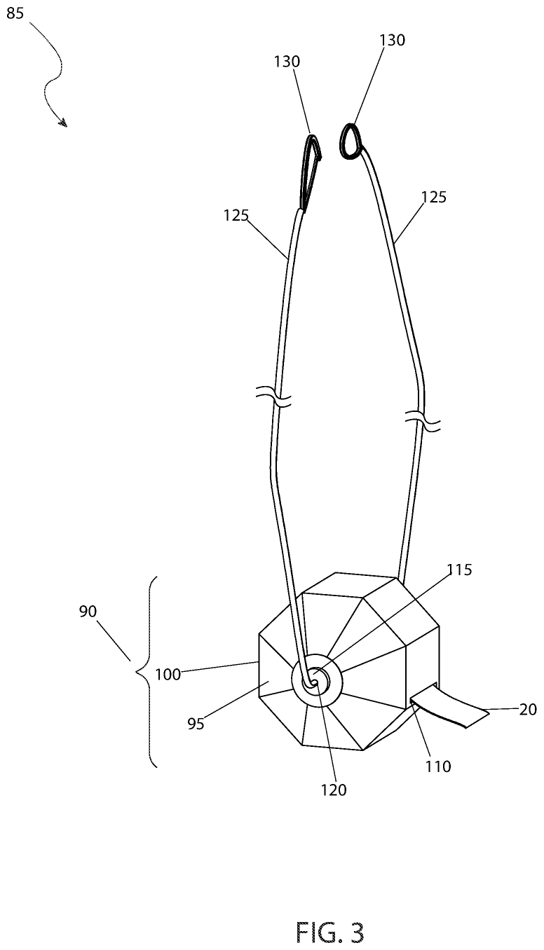

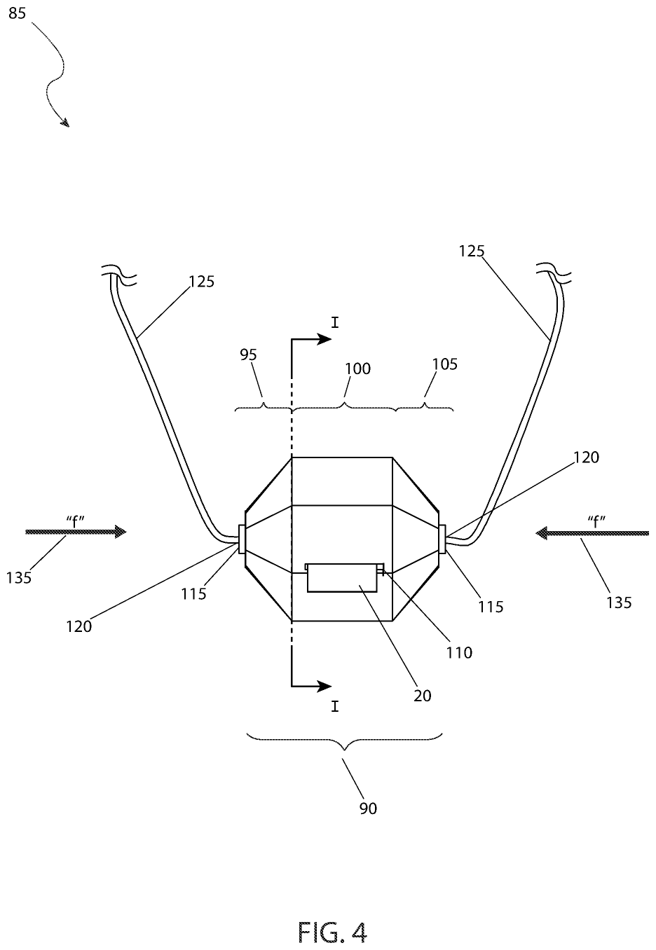

Referring now to FIG. 3, a side perspective view of a tape wrap dispenser 85, used with the CBD-infused therapy tape-and-dispenser 10, according to the preferred embodiment of the present invention is shown. The tape wrap dispenser 85 can be made available in different designs each of which is suitable for the tape 20 (as shown in FIG. 1) to be dispensed. The tape wrap dispenser 85 has a generally spherical outer enclosure 90 which is a twenty-six (26) sided polyhedron. The outer enclosure 90 has nine (9) left side faces 20, eight (8) center section faces 25 (only partially shown in FIG. 1) and nine (9) right side faces 30 (not shown in this figure due to illustrative limitations but see FIGS. 4 through 6). This shape is simply one (1) example of the many possible shapes. However, it does have the advantage of being easily handled. well balanced, symmetrical, while also proving a centered tape exit slot 110. Furthermore, most importantly the outer enclosure 90 has planar sides the enable the tape wrap dispenser 85 to be placed on a flat surface without rolling.

The tape wrap dispenser 85 further includes two (2) diametrically opposed release/cut buttons 35 (only one (1) shown in FIG. 3 but see also FIG. 4). The release/cut buttons 115 are located at the centers of the left side faces 95 and right side faces 105 respectively. It should be understood that the release/cut buttons 115 are internally connected. Further information regarding the release/cut buttons 115 is provided below.

Each release/cut button 115 passes through a center aperture 120. In addition, a neck lanyard 125 is routed through the center aperture 120. The neck lanyard 125 is designed to be worn around the neck of a user such as a coach or trainer. Importantly, the neck lanyard 125 includes a break-away anti-choke quick release mechanism 130 to ensure user safety. By locating the left side and right-side release/cut buttons 115 diametrically opposed to one (1) another simplifies implementing any required mechanical linkages as well as the routing of the neck lanyard 125. Finally, the leading edge of the tape 20 passes through the tape exit slot 55. The balance of the tape roll 15 (as seen in FIG. 1) is retained inside of the tape wrap dispenser 85.

Referring now to FIG. 4, a front view of the tape wrap dispenser 85, used with the CBD-infused therapy tape-and-dispenser 10, according to the preferred embodiment of the present invention is disclosed. The front view shows the generally spherical shape of the outer enclosure 90. The left side faces 95 and the right-side faces 105 are generally trapezoidal while the center section faces 100 are generally rectangular. The planar surfaces are envisioned as being highly useful in preventing or at least resisting the rolling of the tape wrap dispenser 85 when it is set on a flat surface.

FIG. 4 also shows the neck lanyard 125 routed through the center aperture 120 of the release/cut buttons 115. The release/cut buttons 115 are operated by pressing along force lines “f” 135 such as by using a finger and a thumb. Pressing causes a cutting blade 140 (not visible in FIG. 4, due to illustrative limitations) to cut the tape 20 at the point where it leaves the tape exit slot 110 but preferably not all the way through the end of the tape 20. Then, upon release of the release/cut buttons 115, the end of the tape 20 can be pulled to totally shear it way from the remainder of the tape roll 15 (as shown in FIG. 1) while a short segment of the tape 20 is advanced to protrude from the tape exit slot 110 for easy grasping and subsequent future use.

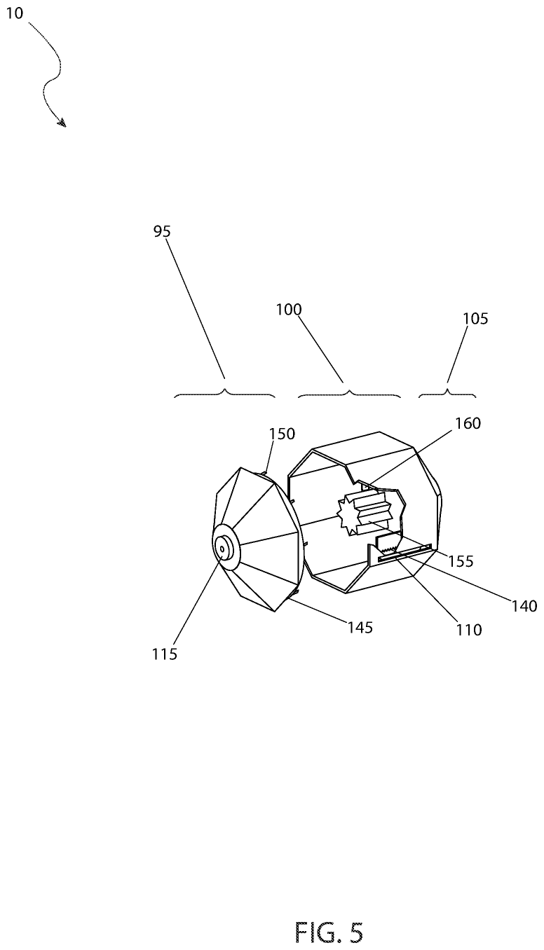

Referring next to FIG. 5, an exploded view of the tape wrap dispenser 85, used with the CBD-infused therapy tape-and-dispenser 10, according to the preferred embodiment of the present invention is depicted. The left side faces 95 are engaged with and connected to the center section faces 100 by a recessed lip 145 having locking tabs 150. When opened as shown in FIG. 5, a central hub 155 that is affixed to the inside of the right side faces 105 is revealed. The central hub 155 is in direct physical contact with the core 25 of the tape roll 15 (both of which are shown in FIG. 1). In practice, the center section faces 100 are permanently fixed to the right side faces 105.

When the release/cut buttons 115 are operated as described above, a linkage cable 160 moves the cutting blade 140 which cuts the tape 20 (as shown in FIG. 1) at the tape exit slot 110 as previously described. For example, the linkage cable 90 may be a “Bowden cable” of the type used for bicycle brakes and gear selection to transmit mechanical power or movement to the cutting blade 140.

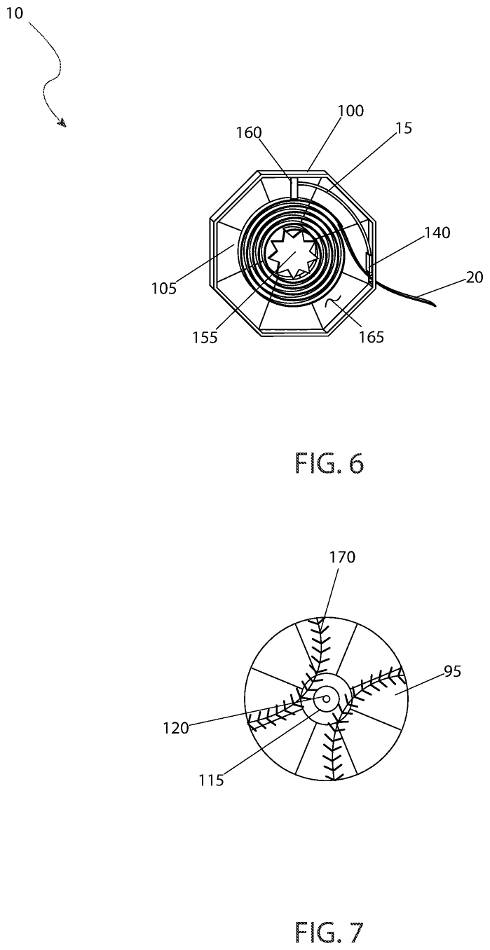

Referring now to FIG. 6, a sectional view of the tape wrap dispenser 85, used with the CBD-infused therapy tape-and-dispenser 10, as seen along a line I-I, as shown in FIG. 4, according to the preferred embodiment of the present invention is shown. The tape roll 15 is shown stowed in position on the central hub 155. The linkage cable 160 is connected to the cutting blade 140. A tape holding cavity 165 is formed by the center section faces 100, the left side faces 95, and the right side faces 105. The tape holding cavity 165 is designed to provide adequate space to hold tape rolls 15 of different widths and diameters.

Referring to FIG. 7, a side view of the tape wrap dispenser 85, having a decorative exterior motif, according to the preferred embodiment of the present invention is disclosed. Various decorative indicia 170 are provided on the left side faces 95 as shown. The decorative indicia 170, here shown as a baseball, may vary per design. Other decorative indicia 170, such that mimics a soccer ball, a tennis ball, or the like are also envisioned. The use of any, none, and all decorative indicia 170 shall not be a limiting factor of the present invention. Other exterior configurations, such as the release/cut buttons 115 and the center aperture 120 remain visible and operable with the decorative indicia 170 present.

### 2. Operation of the Preferred Embodiment

The preferred embodiment of the present invention can be utilized by the common user in a simple and effortless manner with little or no training. It is envisioned that the CBD-infused therapy tape-and-dispenser 10 would be constructed in general accordance with FIG. 1 through FIG. 7. The user would procure the CBD-infused therapy tape-and-dispenser 10 from conventional procurement channels such as CBD distributors, drug stores, pharmacies, mail order and internet supply houses and the like. Special attention would be paid to the physical size of the tape 20, CBD dosage of the tape 20, and any specific exterior decorative indicia 170 of the tape wrap dispenser 85.

After procurement, the tape wrap dispenser 85 would be prepared in the following manner: the left side faces 95 would be disconnected from the center section faces 100 by separating the recessed lip 145 via the locking tabs 150, the desired tape roll 15, including physical size and CBD dosage within the CBD monolayer 80, would be placed on the central hub 155 with the loose end routed of the tape 20 passing through the tape exit slot 110; the left side faces 95 would be reattached to the center section faces 100; the neck lanyard 125 would be routed through the center aperture 120; and, the anti-choke quick release mechanism 130 would be engaged about the user's neck.

During use, the outer enclosure 90 would be grasped in one (1) hand by the user; the user would then grab the leading edge of the tape 20 and pull the tape 20 from the tape wrap dispenser 85 until the tape 20 has the desired length; both release/cut buttons 115 would be pressed by the user by applying force along force lines “f” 135 using a finger and thumb on one (1) hand while the other hand pulls the now cut leading edge of the tape 20 from the remainder of the CBD infused tape roll 15. The section of tape 20 is then applied to the desired skin area of the user to produce desired pain relief effects.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated.

What is claimed is:

1. A CBD-infused therapy tape and dispenser device for ease and mobile convenience for a person requiring the need of CBD for medical purposes, the device comprising:
a tape, the tape having a middle, an interior surface and an exterior surface, the tape infused with cannabidiol (CBD) solution; and
a core, the core being in a middle of a tape roll when the tape is rolled around the core.

2. The device according to claim 1, wherein the tape roll is configured with a leading edge, wherein the leading edge is a linear portion of the tape.

3. The device according to claim 1, wherein the core is disposable.

4. The device according to claim 1, wherein the exterior surface of the tape is water resistant.

5. The device according to claim 1, wherein the tape is kinesiology tape.

6. The device according to claim 1, wherein the interior surface of the tape has an adhesive coating.

7. The device according to claim 7, wherein the adhesive coating is a skin-safe material.

8. The device according to claim 1, wherein CBD solution comprises two grams of CBD isolate dissolved in ninety-nine percent isopropyl alcohol.

9. The device according to claim 1, wherein the CBD solution contains twenty-five milligrams per milliliter.

10. The device according to claim 1, wherein the tape is rolled onto a tape roll dispenser, wherein the tape roll dispenser has two diametrically opposed release/cut buttons.

11. The device according to claim 10, wherein the release/cut buttons are coupled internally in the tape roll dispenser.

12. A method of making a CBD-infused tape roll, the method comprising:
unrolling the tape roll, wherein the unrolled tape roll presents an interior surface;
formulating a CBD solution;
using an applicator to apply the CBD solution onto the raw tape roll onto the interior surface of the unrolled tape roll.

13. The method according to claim 12, wherein the applicator is a roll-on-applicator.

14. The method according to claim 13, wherein the roll-on-applicator is a roller ball.

15. The device according to claim 12, wherein CBD solution comprises two grams of CBD isolate dissolved in ninety-nine percent isopropyl alcohol.

16. The device according to claim 12, wherein storing the CBD solution in a reservoir.

17. The device according to claim 16, wherein routing the CBD solution to the applicator.

18. The method of claim 17, wherein the applicator is a spray mechanism.

19. The device according to claim 17, wherein the spray mechanism has a spray nozzle.

20. The device according to claim 18, wherein spraying the CBD solution by an aerosol spray from the spray nozzle to the interior surface of the tape, and wherein after the CBD solution has dried on the interior surface of the tape, rewind the tape into a tape roll.
